(19) United States
(12) Patent Application Publication
(10) Pub. No.: US 2025/0061981 A1
(43) Pub. Date: Feb. 20, 2025

(54) MINERALIZATION SIMULATION IN THE MICROSCALE

(71) Applicant: International Business Machines Corporation, Armonk, NY (US)

(72) Inventors: JAIONE TIRAPU AZPIROZ, Rio de Janeiro (BR); David Alejandro Lazo Vasquez, São Paulo (BR); Rodrigo Neumann Barros Ferreira, Rio de Janeiro (BR); Mathias B Steiner, Rio de Janeiro (BR)

(21) Appl. No.: 18/233,508

(22) Filed: Aug. 14, 2023

(51) Int. Cl. G16C 60/00; G06F 30/27; G06F 30/28

(52) U.S. Cl. CPC G16C 60/00; G06F 30/27; G06F 30/28; G06F 2113/08

(57) ABSTRACT

A grey-scale tomography of a sample is obtained and a capillary network model (CNM) geometry of the sample is produced, using a capillary network extractor, based on the grey-scale tomography. A fluid flow simulation is performed, using a flow simulator, to generate flow property fields based on the capillary network model (CNM) geometry. A mineralization simulation is performed influenced by results of the flow property fields to generate a modified capillary geometry that represents the capillary network model (CNM) geometry at a future point in time.

### BACKGROUND

The present invention relates generally to the electrical, electronic and computer arts and, more particularly, to computer-aided materials science, computational fluid mechanics, and the like.

Mineralization simulation methods in porous media mostly rely on performing numerically intensive computations of chemical and physical reactions on small portions of the structure. These methods use high-resolution mesh-like representations of the pore geometry and solve a system of transport-reaction differential equation systems through numerical methods (that is, finite differences, finite volume, or finite element methods). Conventional solutions do not solve transport reaction on capillary network representations of the rock pore space, or address the interplay between the evolution of the geometry and the progression of the reaction of simultaneous physical and chemical processes within the capillary network. Consequently, conventional methods do not address the effect of simulating vastly different time scales. Even when more efficient representations of the porous geometry, like pore or capillary networks, are used, mineralization simulations tend to be restricted to the effects of the pore-scale process. Most formulations target a single fluid-solid process in the capillary per iteration. When including two or more processes, these conventional formulations fail to adjust the temporal intervals to consider the effects of different thresholds for the onset of different processes and their influence on fluid-solid interactions and geometry evolution at each capillary in time, i.e. the conventional formulations apply the smallest relevant time scale to the simulation of all processes under consideration.

### BRIEF SUMMARY

Principles of the invention provide systems and techniques for mineralization simulation in the microscale. In one aspect, an exemplary method includes the operations of obtaining a grey-scale tomography of a sample; producing, using a capillary network extractor, a capillary network model (CNM) geometry of the sample based on the grey-scale tomography; performing, using a flow simulator, a fluid flow simulation to generate flow property fields based on the capillary network model (CNM) geometry; and performing a mineralization simulation influenced by results of the flow property fields to generate a modified capillary geometry that represents the capillary network model (CNM) geometry at a future point in time.

In one aspect, a computer program product comprises one or more tangible computer-readable storage media and program instructions stored on at least one of the one or more tangible computer-readable storage media, the program instructions executable by a processor, the program instructions comprising obtaining a grey-scale tomography of a sample; producing, using a capillary network extractor, a capillary network model (CNM) geometry of the sample based on the grey-scale tomography; performing, using a flow simulator, a fluid flow simulation to generate flow property fields based on the capillary network model (CNM) geometry; and performing a mineralization simulation influenced by results of the flow property fields to generate a modified capillary geometry that represents the capillary network model (CNM) geometry at a future point in time.

In one aspect, an apparatus comprises a memory and at least one processor, coupled to the memory, and operative to perform operations comprising obtaining a grey-scale tomography of a sample; producing, using a capillary network extractor, a capillary network model (CNM) geometry of the sample based on the grey-scale tomography; performing, using a flow simulator, a fluid flow simulation to generate flow property fields based on the capillary network model (CNM) geometry; and performing a mineralization simulation influenced by results of the flow property fields to generate a modified capillary geometry that represents the capillary network model (CNM) geometry at a future point in time.

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor, oil field equipment, carbon dioxide sequestration equipment, or the like, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects. Some embodiments may not have these potential advantages and these potential advantages are not necessarily required of all embodiments. By way of example only and without limitation, one or more embodiments may provide one or more of:
techniques for computing the impact of fluid-rock processes on the pore scale geometry;
a computational workflow that enables the prediction, within a capillary network representation framework of a rock sample, the spatiotemporal geometry evolution of the porous structure resulting from pore-scale processes;
a system for estimating the volume of carbon dioxide converted and stored at a large scale, such as on the scale of a reservoir, based on pore-scale processes;
techniques that address the interplay between changes in the local flow conditions of a porous structure due to geometry evolution and their influence on the physical and chemical reactions;
techniques that consider the influence of the local flow conditions at the onset and the evolution of each process (for example, the local flow rate can influence the onset and rate of erosion);
techniques that address the progression of simultaneous physical and chemical processes within the capillary network of a porous material and the effect of simulating vastly different time scales;
techniques for optimizing the simulation parameters for maximum efficiency while retaining accuracy;
techniques for adopting the reaction formulations to the capillary network representation enabling larger rock sample volumes in simulation with same computing resources;
improving a variety of practical applications, such as reservoir engineering, oil recovery, carbon dioxide geological sequestration, and the like; and
supporting the generation of new additive candidates through simulation to accelerate mineralization under geological conditions.

These and other features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1 is a high-level block diagram for computing the impact of fluid-rock processes on the pore scale geometry, in accordance with an example embodiment;

FIG. 2 is a high-level block diagram of a system for generating a revised capillary network model (CNM) geometry for a future point in time relative to a given capillary network model (CNM) geometry, in accordance with an example embodiment;

FIG. 3A is a three-dimensional (3D) visualization of the evolution of the geometry produced by the geometry modifications module, displaying the CNM representation of the pore space geometry of a small sandstone rock sample at two time steps, in accordance with an example embodiment;

FIG. 3B is a histogram of the distribution of the capillary diameter for the revised version of the capillary network model (CNM) geometry produced by the geometry modifications module of the same sandstone rock sample at two time steps, in accordance with an example embodiment;

FIG. 4 is a flowchart for an example iterative method to simulate fluid flow and multiple physical processes and chemical reactions at pore scale, in accordance with an example embodiment;

FIG. 5A is a table containing exemplary flow conditions, phasic properties, pore-scale process parameters and geometry of the capillary network used for simulating the effects of erosion, in accordance with an example embodiment;

FIG. 5B is a three-dimensional (3D) visualization of a capillary network model of the pore space of the rock sample to be used for the simulation represented as a network of cylindrical capillaries color-coded in terms of the capillary diameter, in accordance with an example embodiment;

FIG. 5C is a three-dimensional (3D) visualization of a capillary network pressure field, in accordance with an example embodiment;

FIG. 6A is a three-dimensional (3D) visualization of the distribution of potential onset of erosion at the initial conditions where the capillaries with a value of one have the highest probability of erosion occurs, leading to a morphology evolution in that site after a simulation time step, in accordance with an example embodiment;

FIG. 6B is a three-dimensional (3D) visualization of the morphology changes of a rock sample under calcite erosion at three different time steps, in accordance with an example embodiment;

FIG. 6C is a two-dimensional (2D) plot of the evolution of the rock sample porosity under calcite erosion as computed after each simulation iteration (showing a gradual increase in porosity as the rock surface is being eroded and the capillary diameters increase), in accordance with an example embodiment;

FIG. 7 is a flowchart of an example method for determining the initial boundary and conditions, in accordance with an example embodiment;

FIG. 8 is a flowchart of an example method for determining the thresholds for the onset of mineral reactions, in accordance with an example embodiment;

FIG. 9 is a flowchart of an example method for computing the combined time-scale for the simulation, in accordance with an example embodiment;

FIG. 10 is a flowchart of an example method for generating the geometry modification due to coupled phenomena, in accordance with an example embodiment;

FIG. 11 is a flowchart of an example method for determining a completion time of a mineralization simulation, in accordance with an example embodiment;

FIG. 12A illustrates the mathematical equations for quantifying the capillary radius increase resulting from erosion and dissolution, in accordance with an example embodiment;

FIG. 12B illustrates the mathematical equations for quantifying the capillary radius reduction resulting from deposition and precipitation, in accordance with an example embodiment;

FIG. 13 illustrate the parameters of the simultaneous pore-scale processes, in accordance with an example embodiment;

FIG. 14A illustrates a timing diagram for performing a mineralization simulation, in accordance with an example embodiment;

FIG. 14B is an illustrative graph of the diameter of each capillary, in accordance with an example embodiment; and

FIG. 15 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

Generally, techniques are disclosed for computing the impact of fluid-rock processes on the pore scale geometry. In one example embodiment, a cloud-based simulation toolkit is provided for computing the simulation. Example embodiments have applications in reservoir engineering, oil recovery, carbon dioxide geological sequestration, and the like.

In one example embodiment, a computational workflow enables the prediction, within a capillary network representation framework of a rock sample, the spatiotemporal geometry evolution of the porous structure resulting from pore-scale processes associated with, for example, subsurface carbon dioxide (CO₂) injection and sequestration (such as erosion, deposition, dissolution, precipitation, and the like). The geometrical changes are then used, for example, to estimate the volume of carbon dioxide (CO₂) converted and stored at a large scale, such as on the scale of a reservoir.

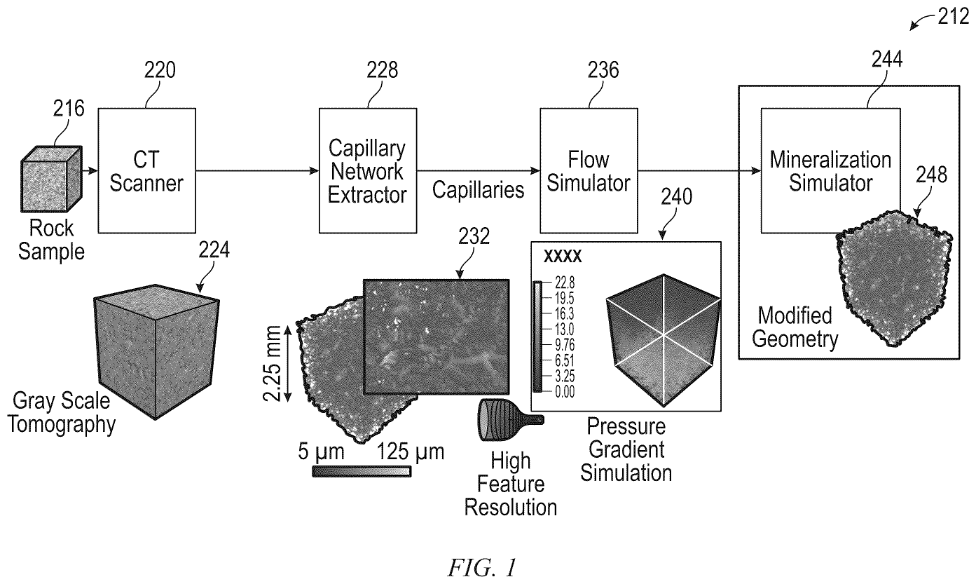

FIG. 1 is a high-level block diagram 212 for computing the impact of fluid-rock processes on the pore scale geometry, in accordance with an example embodiment. In one example embodiment, a computerized tomography (CT) scanner 220 scans a rock sample 216 to generate a grey-scale tomography 224. (A scan may include, for example, one million to one billion volume elements or voxels.) A capillary network extractor 228 produces a capillary network model (CNM) geometry 232 of the capillaries of the rock sample 216 based on the grey-scale tomography 224. For example, image processing may be used to convert the grey-scale of the scan to binary values, to sharpen the captured image, and the like. A flow simulator 236 then performs a fluid flow simulation where flow is driven by a pressure gradient applied across the width of the sample 216 in one of its axes to generate pressure gradient field 240, flow rate field and the like based on the capillary network model (CNM) geometry 232. To simulate both single and two-phase flow through the capillary network model of the pore geometry, laminar flow is assumed and the equations relating pressure and flow are applied within each capillary, followed by conservation of mass at each network node, to build a large system of coupled equations in sparse matrix form. Solving this matrix yields flow properties, like the pressure distribution or flow rate, at each point in the network, from where to extract bulk flow properties like permeability or saturation by one fluid. A mineralization simulator 244 runs a simulation based on the pressure gradient results of the flow simulation to generate a modified capillary geometry 248 that represents the capillary network model (CNM) geometry 232 at a future point in time, such as after a period of precipitation, erosion, and the like.

### Simulating the Impact of Precipitation

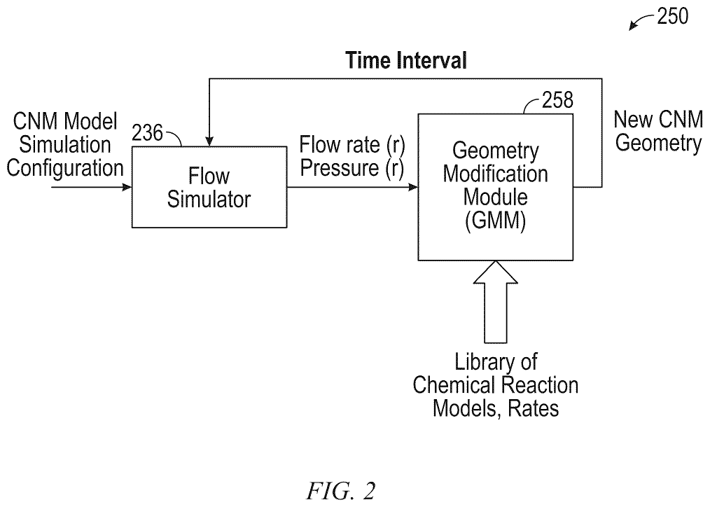

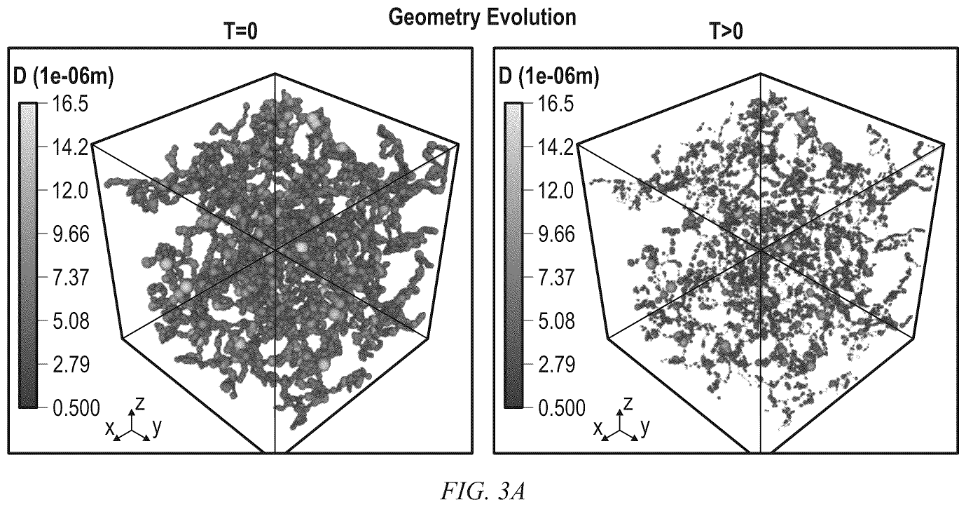

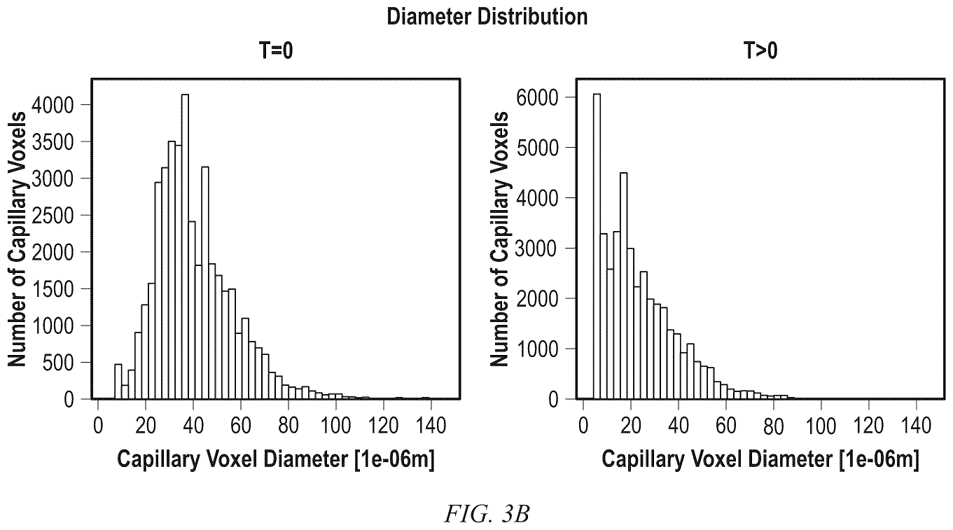

In one or more embodiments, the evolution of the porous geometry due to fluid-rock interactions is modeled by estimating changes in the capillary diameter through an iterative computation over time. FIG. 2 is a high-level block diagram of a system 250 for generating a revised capillary network model (CNM) geometry 248 for a future point in time relative to a given capillary network model (CNM) geometry 232, in accordance with an example embodiment. In one example embodiment, the flow simulator 236 produces metrics such as flow rate (r), pressure (r), and the like based on a specified capillary network model (CNM) simulation configuration. A geometry modifications module 258 of the mineralization simulator 244 produces, using a library of chemical reaction models, rates, and the like, a revised capillary network model (CNM) geometry based on the produced metrics. FIG. 3A is a three-dimensional (3D) visualization of the evolution of the geometry produced by the geometry modifications module 258, displaying the CNM representation of the pore space geometry of a small sandstone rock sample 216 at two time steps, in accordance with an example embodiment. One time step occurs at the beginning of the simulations (T=0; left-side CNM representation) and one time step occurs after a few simulation iterations have elapsed (T>0; right-side CNM representation). FIG. 3B is a histogram of the distribution of the capillary diameter for the revised version of the capillary network model (CNM) geometry 232 produced by the geometry modifications module 258 of the same sandstone rock sample 216 at the same two time steps, as produced by the geometry modifications module 258, in accordance with an example embodiment.

### Conventional Scenario

Conventional mineralization simulation methods in porous media rely mostly on performing numerically intensive computations of fluid flow, mineral concentration distribution, and chemical reactions within the simulation structure. These methods generally use high-resolution mesh-like representations of the pore geometry and solve a system of transport-reaction differential equation systems through numerical methods (i.e., finite differences, finite volume, or finite element methods). As such, these methods are limited to small simulation domains of the extent of a few micrometers or a few pores, and demand high computational resources to extend to simulation volumes on the order of a representative element volume (REV) of the rock sample 216. Similarly, conventional systems do not provide for the simulation of both chemical and physical processes simultaneously considering distinct time scales, and fail to adjust the simulation time steps to the process-specific time-scales.

An exemplary flow simulation toolkit, according to aspects of the invention, relies on a sparse graph representation of the pore geometry of the rock sample 216, i.e. a capillary network model, with reduced degrees of freedom as compared with mesh-or lattice-based methods, enabling fast fluid flow simulations even for large samples 216 with millions of capillaries. Conventional mineralization simulation solutions do not address the interplay between changes in the local flow conditions due to the geometry evolution and their influence on the reaction. Even when more efficient representations of the porous geometry, like pore or capillary networks, are used, conventional mineralization simulations are restricted to the effects of the pore-scale process; in other words, conventional mineralization simulations do not consider the influence of the local flow conditions on the onset and evolution of each process (for example, the local flow rate that can influence the onset and rate of erosion). Conventional mineralization simulations also do not address the progression of simultaneous physical and chemical processes within the capillary network and the effect of simulating vastly different time scales.

Example embodiments simulate the impact of fluid-rock interactions in the pore space of a porous rock under the framework of a capillary network representation of that pore space by estimating changes in each capillary diameter that incorporate the influence of local fluid flow conditions on the reaction rates at each temporal iteration. The iterative computation also accommodates for multiscale phenomena with very different time scales by determining, at each iteration, which processes to compute based on their characteristic reaction time. For example, precipitation may be simulated for future times separated by many time steps (for example, 2,000 seconds with iterations of 500 seconds).

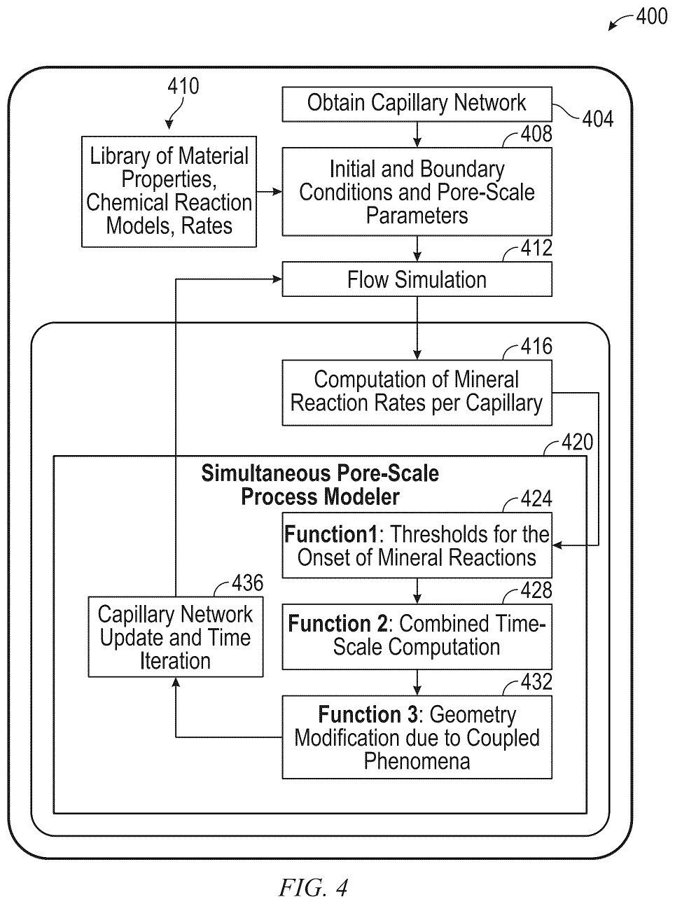

FIG. 4 is a flowchart for an example iterative method 400 to simulate fluid flow and multiple physical processes and chemical reactions at pore scale, in accordance with an example embodiment. For example, subsurface CO₂ injection and sequestration may be simulated. In one example embodiment, a capillary network model (CNM) geometry 232 (also referred to as a capillary network representation herein) of the porous rock sample 216 is obtained from, for example, the capillary network extractor 228 (operation 404). The grayscale volume of the digitalized rock obtained from the μCT (X-ray micro-computed-tomography) measurements is processed with an appropriate thresholding algorithm and segmented into solid regions and void spaces, leading to a binary image where black voxels represent void or pore space and white voxels represent solid rock matrix. The rock pore space geometry is then modeled as a network of connected capillaries or short cylinders, each of the length of one voxel, and with spatially varying radius to match the local geometry extracted from the microtomography. Starting from the high-resolution binary image, the most central path from inlet pore to outlet pore of each set of connected pore space voxels is extracted and saved into a graph. The resulting graph is used to build a network in which every graph node becomes a cylindrical capillary with diameter computed to minimize the distance to the pore boundary and with the connection to adjacent capillaries saved as node properties. Initial and boundary conditions and pore-scale parameters are determined (operation 408). For example, based on the materials and selected reactions, phasic properties, flow conditions, and mineral reaction parameters are determined. The initial and boundary conditions may be based on pore-scale process parameters, such as datasets of experimental parameters collected from the literature, stored in dedicated library 410. The global initial and boundary conditions determined in step 408 include those needed for the flow simulation 412, such as fluid properties (e.g. viscosity), flow conditions (e.g. absolute temperature and driving pressure gradient across the rock sample 216), and those needed for the pore-scale process modeler 420 (such as reaction parameters, including reaction formulation of choice, reaction rates, total simulation time, and the like). Fluid-flow numerical simulations in the capillary network are performed (operation 412). Per iteration, certain boundary conditions used in the pore-scale process modeler 420, like the distribution of pressure and flow rate within the network, are extracted from the results of the flow simulator 412.

In one example embodiment, operations 412-436 are repeated until a steady state or stopping criterion is reached. For example, operations 412-436 may be repeated until the porosity of the rock sample 216 reaches a given level. The final porosity is calculated considering a relationship between the initial porosity and the variation of the capillary distribution. The model performs simulations until the capillary diameters reach minimum or maximum values pre-determined, for example, in the code. For example, for mineral precipitation simulations, the minimum capillary diameter is 0.5 voxels. Simulations may be stopped when a prefixed number of iterations is reached, when porosity computations between successive iterations reveal changes smaller than a predetermined criterion (e.g., below a certain percentage change between steps), or when the latest porosity value is within a predetermined margin of a value set in step 408. Given the teachings herein, the skilled person can heuristically determine a suitable percentage change and/or predetermined margin and set value, for a given domain of interest and set of conditions.

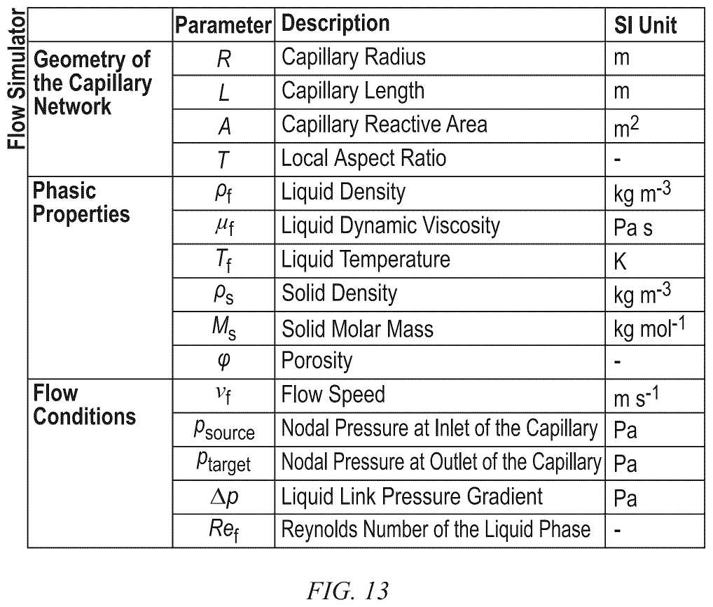

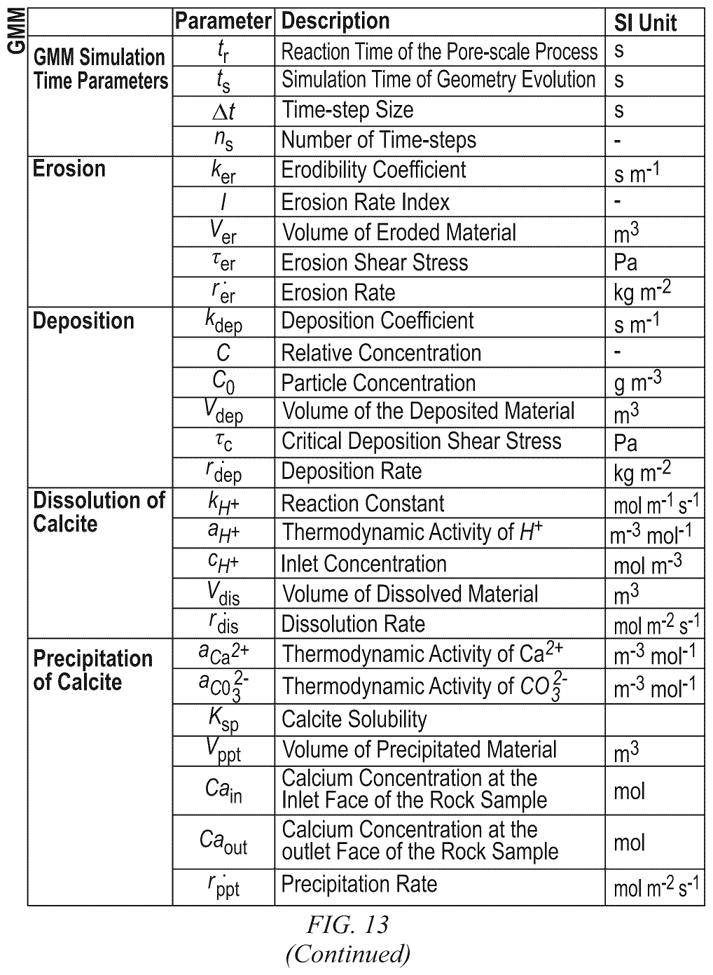

Mineral (physical and chemical) reaction rates per capillary are computed (operation 416). The physical and chemical reaction rates are modeled with correlations from the literature, consisting of fluid and particle dynamics expressions and phenomenological models. The parameters related to the calculation of physical and chemical reaction rates are detailed in FIG. 13. The rates and thresholds corresponding to physical pore-scale processes involving calcite and brine are defined by a known erosion and deposition law:

$${r}_{er , dep}^{\cdot } = \{ \begin{matrix}- {κ}_{er} ( {τ}_{w} - {τ}_{er} ) & {τ}_{w} > {τ}_{er} \\ + C {κ}_{dep} ( {τ}_{dep} - {τ}_{w} ) & {τ}_{w} < {τ}_{dep} \\ 0 & {τ}_{er} \geq {τ}_{w} \geq {τ}_{dep}\end{matrix}$$

Alternately, mineral dissolution and precipitation rates of calcite in brine are calculated with phenomenological correlations, based on affinity models of surface-controlled crystallization. Dissolution rates are defined by a known correlation:

$${r}_{dis} = {k}_{H +} {γ}_{H +} {c}_{H +}$$

Precipitation rates are defined by a known correlation:

$${r}_{ppt = - Q \frac{{Ca}_{out} - {Ca}_{i n}}{A}}$$

The thresholds for the onset of the chemical processes are defined following known criteria, in which, after a short delay (while calcium ions are convected towards the outlet), the average reaction rate increases sharply with time, leading to significant morphology changes. For example, two seconds for dissolution and 1200 seconds for precipitation. In sum, following known procedures, the flow simulator calculates the fluid flow fields until the fluid reaches a steady state, before turning on the pore-scale processes. The reaction times necessary for significant average reaction rates to take place are defined by experimental data from the literature.

A simultaneous pore-scale process modeler 420 then performs operations 424 to 432 to generate an update for the capillary network model (CNM) geometry 232 to be used by the flow simulator 236 during a subsequent iteration. In one example embodiment, thresholds for the onset of mineral reactions are determined (operation 424). For example, the existence of relevant pore-scale process(es), such as erosion and precipitation, at each capillary are identified depending on thresholds. The thresholds may be based, for example, on the known values in the literature. The most suitable reaction time periods for updating the capillary network model (CNM) geometry 232 and performing another flow simulation are determined based on the distinct time scales of the process per capillary (operation 428). For example, the optimal frequency for performing a simulation for a given effect, such as erosion, precipitation, and the like, are determined. In one example embodiment, operations 424 and 428 may be skipped and the frequency of the simulation for the given effect may be set to a predefined value. The frequency of turning on the simulation of each pore-scale process depends on their timescale. During a simulation cycle, if two or more processes are considered, the reaction times are classified from the lowest to the highest. The geometry variation of the process with the lowest reaction times is simulated iteratively until reaching the second lowest reaction time, and then the next process simulation is turned on. This process is repeated iteratively until reaching the longest reaction time, completing one cycle. The simulation stop criteria is defined as an input final simulation time.

The geometry modification due to the coupled phenomena, such as erosion, precipitation, and the like, is generated (operation 432). For example, with information about the time intervals for performing a simulation for each effect and the reaction rates, the change in diameter of each capillary is computed. The geometry of the capillary network is iteratively updated with each pass through the method 400 based on the individual contributions of each selected effect to produce the capillary network update of the capillary network update and time iteration operation (operation 436). The method 400 continues until the simulation criteria is satisfied.

### Example Use Case: MVP Onset of Erosion

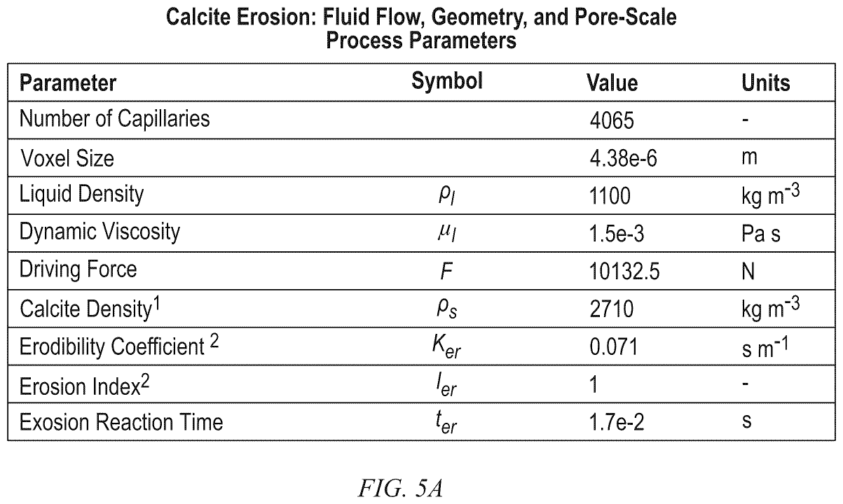

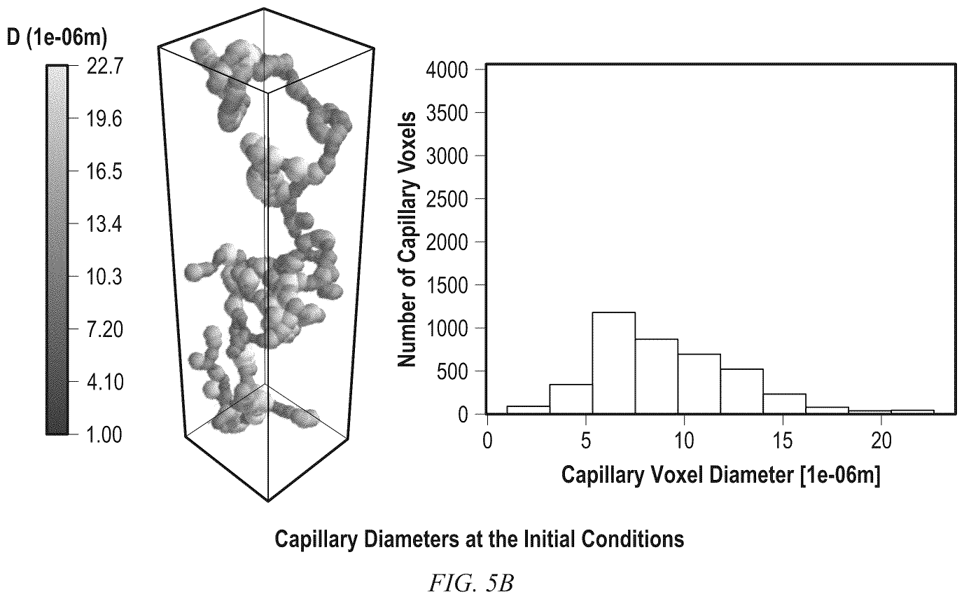

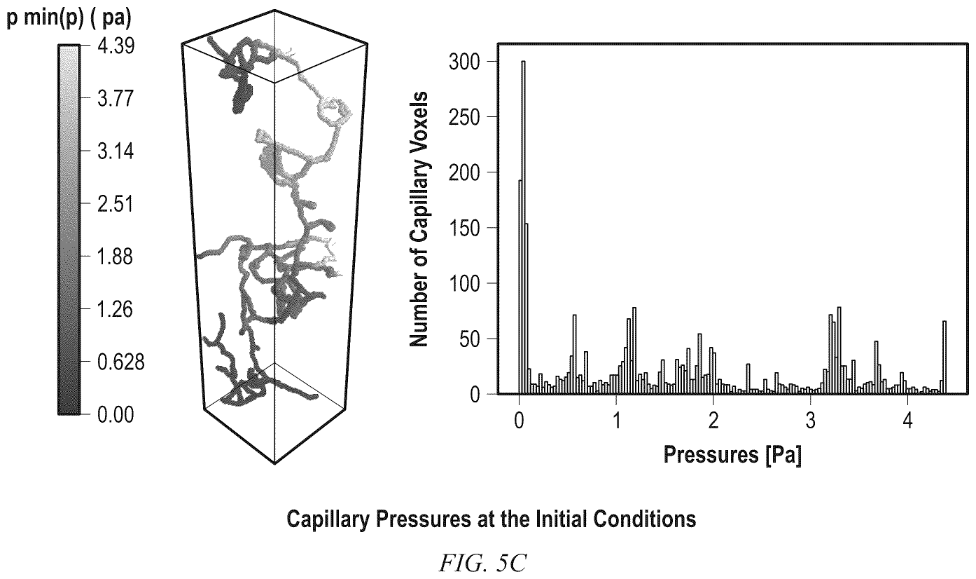

FIG. 5A is a table containing exemplary flow conditions, phasic properties, pore-scale process parameters and geometry characteristics of the capillary network used for simulating the effects of erosion, in accordance with an example embodiment. Parameters ‘number of capillaries’ and ‘voxel size’ in FIG. 5A refer to the capillary network properties; parameters ‘liquid density’, ‘dynamic viscosity’ and ‘driving force’ refer to fluid properties and flow boundary conditions, respectively; parameter ‘liquid density’ (as well as parameters ‘erodibility coefficient’ and ‘erosion index’) refer to rock and fluid properties necessary in the computation of the erosion reaction impact on the surface of the capillary.

FIG. 5B is a three-dimensional (3D) visualization of the capillary network model of the pore space of the rock sample 216 to be used for the simulation as extracted by the capillary network extractor 228 and represented as a network of cylindrical capillaries, coded in terms of the capillary diameter, in accordance with an example embodiment. A histogram depicts the number of capillary voxels for a range of capillary voxel diameters. FIG. 5C is a three-dimensional (3D) visualization of the pressure field on the same capillary network as FIG. 5B as computed by the flow simulator 236 in the flow simulation step 412, in accordance with an example embodiment.

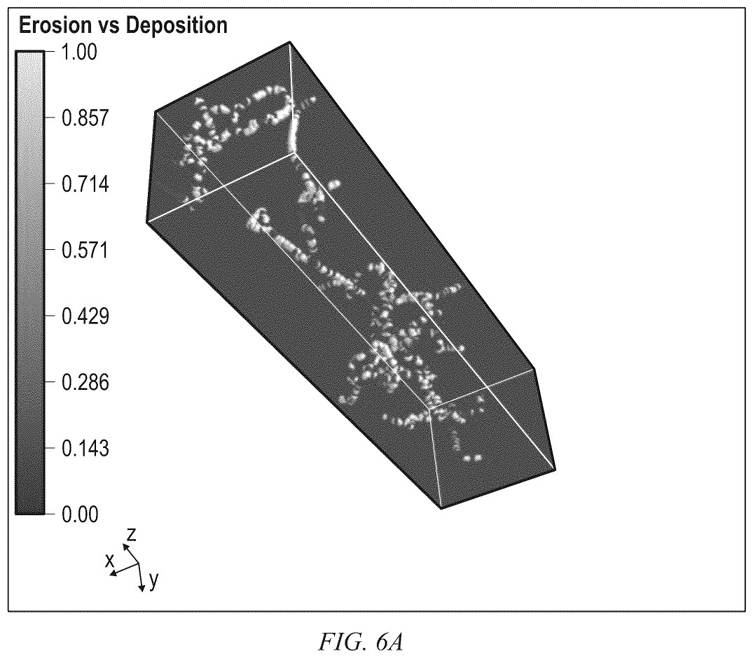

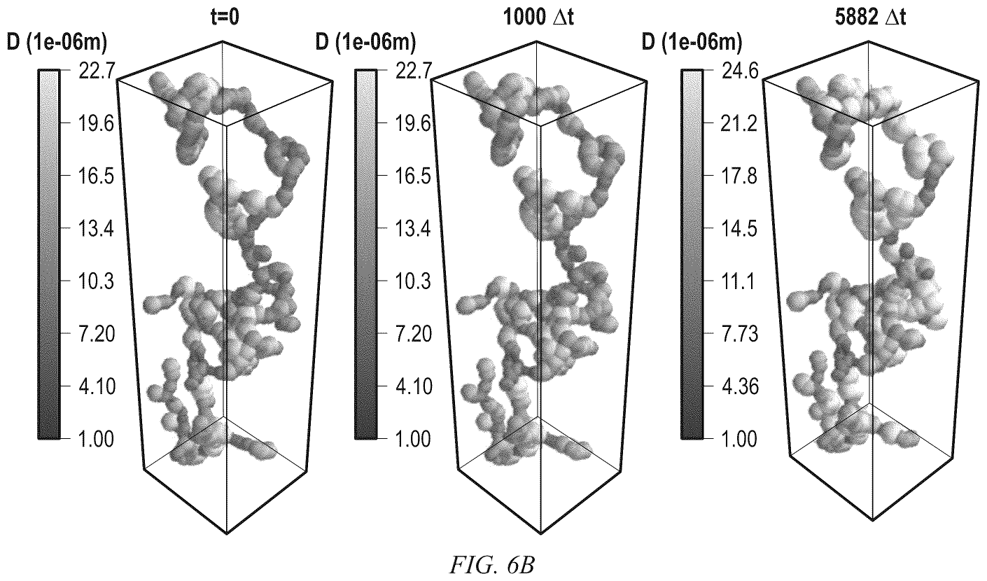

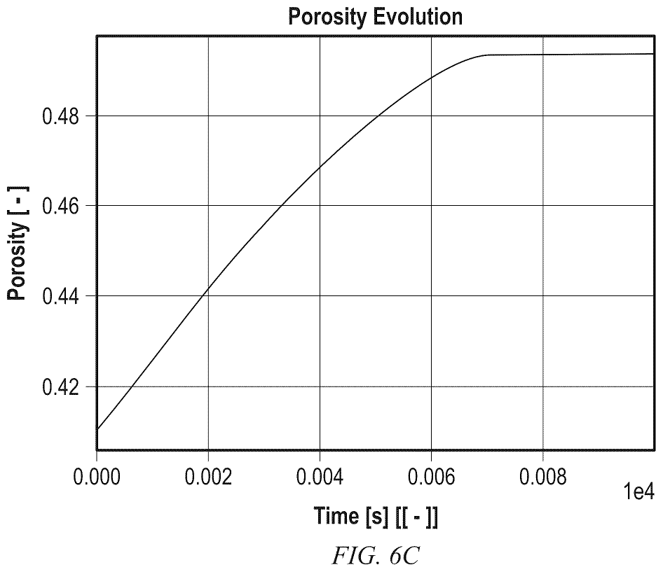

FIG. 6A is a three-dimensional (3D) visualization of the distribution of potential onset of erosion at the initial conditions where the capillaries with a value of one have the highest probability of erosion occurring, leading to a morphology evolution in that site after a simulation time step, and the capillaries with number 0 display the smallest probability of erosion occurring at that capillary and thus, experiencing no morphology changes in that iteration, in accordance with an example embodiment. In FIG. 6A, the number one stands for capillaries where erosion occurs, according to the thresholds of a known erosion and deposition law.

FIG. 6B is a three-dimensional (3D) visualization of the morphology changes of a rock sample 216 under calcite erosion at three different time steps, in accordance with an example embodiment. One time step is at the beginning of the simulations (T=0), one time step is after 1000 time steps (1000 Δt) and one time step is after 5882 time steps have elapsed (5882 Δt), thereby displaying an expansion of the pore space as result of the rock 216 being etched due to the erosion reaction being simulated.

FIG. 6C is a two-dimensional (2D) plot of the evolution of the rock sample porosity under calcite erosion as computed after each simulation iteration (showing a gradual increase in porosity as the rock surface is being eroded and the capillary diameters increase), in accordance with an example embodiment. The increase in porosity value reaches a saturation level after a number (in this example, 5882) of iterations, indicating that the changes in the pore space geometry induced changes in the flow conditions and reaction rate values that diminished the impact on the rock surface in time.

### Initial Boundary and Conditions

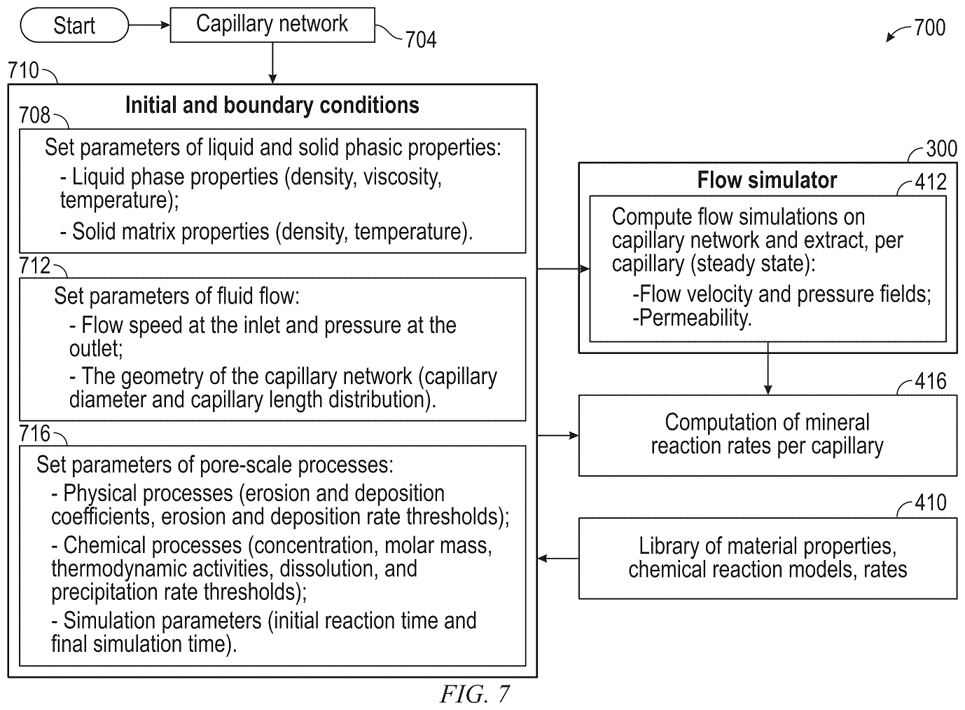

FIG. 7 is a flowchart 700 of an example method for determining the simulation initial and boundary conditions, in accordance with an example embodiment. In one example embodiment, a capillary network is obtained (operation 704). Given a rock sample 216, a file containing the information on the geometry of each capillary of the network, as extracted by the capillary network extractor 228, is used as the geometrical basis for the simulations. Parameters related to phasic properties, fluid flow and pore-scale processes are selected in step 710 from a dataset library of material properties, chemical reaction models and rates 410 to be imposed as initial and boundary conditions for the simulations. In particular, the parameters of liquid and solid phasic properties are set (operation 708). For example, the liquid phase properties (density, viscosity, and temperature) and the solid matrix properties (density and temperature) are set.

The parameters of fluid flow are set (operation 712). For example, the flow speed at the inlet and pressure at the outlet, and the geometry of the capillary network (capillary diameter and capillary length distribution) are set.

The parameters of the pore-scale processes are set (operation 716). For example, the physical processes (erosion and deposition coefficients, erosion and deposition rate thresholds, and the like), the chemical processes (concentration, molar mass, thermodynamic activities, dissolution, and precipitation rate thresholds), and the simulation parameters (initial reaction time and final simulation time) are set. The literature presents several sets of parameters corresponding to the results of numerical simulations combined with experimental validation. A library of formulations developed from correlating to these experimental results and the associated variables used in those formulations, as well as the ranges of values that those variables operate on, is collected and stored in dataset 410 and used to select the appropriate values for the simulations.

The flow simulations on the capillary network are computed and extracted per capillary (at steady state) by the flow simulator 236 (operation 412). For example, flow velocity and pressure fields, and permeability are computed. Mineral reaction (physical and chemical) rates per capillary are computed by the mineralization simulator 244 (operation 416).

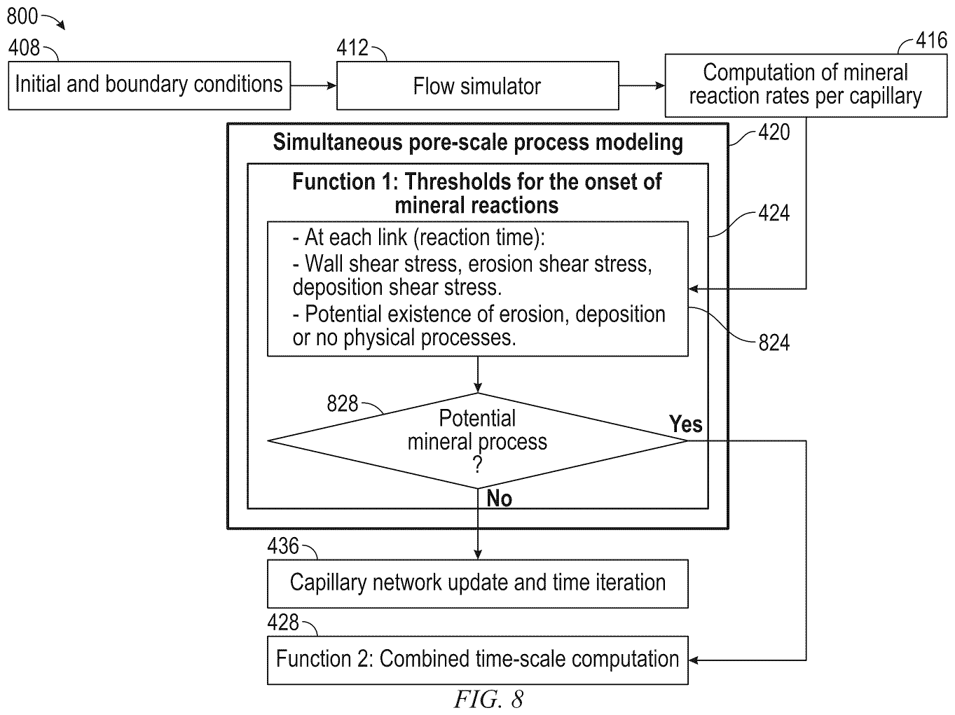

FIG. 8 is a flowchart of an example method 800 for determining the thresholds for the onset of mineral reactions, in accordance with an example embodiment. In one example embodiment, once the initial and boundary conditions are set (operation 408), the flow simulation is run (operation 412), and the mineral reaction (physical and chemical) rates per capillary are computed by the mineralization simulator 244 (operation 416) considering the influence of the flow conditions, like flow rate and pressure gradient in each capillary, in the reaction rates computation. Additional pore-scale process parameters are specified next. In particular, in Function 1 (operation 424) of FIG. 4, the thresholds for the onset of mineral reactions are determined. The potential onset of physical processes is determined for each capillary in the capillary network model (CNM) geometry 232 as they depend on the local fluid flow and geometrical conditions within the pore, such that the geometric evolution rate can vary from capillary to capillary within the entire network. Thresholds refer to certain flow conditions or minimum reaction times inherent to each mineral process that need to be exceeded for a reaction to take place. Examples of thresholds include minimum reaction times required for a detectable volume of mineral within the simulation spatial resolution to be produced via, for instance, a deposition process or removed via, for instance, a dissolution process, and/or a minimum shear stress value computed from flow conditions, like flow speed, that can induce a physical effect like erosion. In other words, phenomenological correlations for chemical processes based on mineral rates, and of physical processes based on the erosion and deposition law are used to compute the onset of mineral reactions. In particular, at each capillary after each iteration, wall shear stress, erosion shear stress, and deposition shear stress are computed to determine the potential existence of erosion, deposition or no physical process at all during operation 824. Also, during operation 824, the computed reaction rate per process is compared to the minimum needed to be spatially relevant.

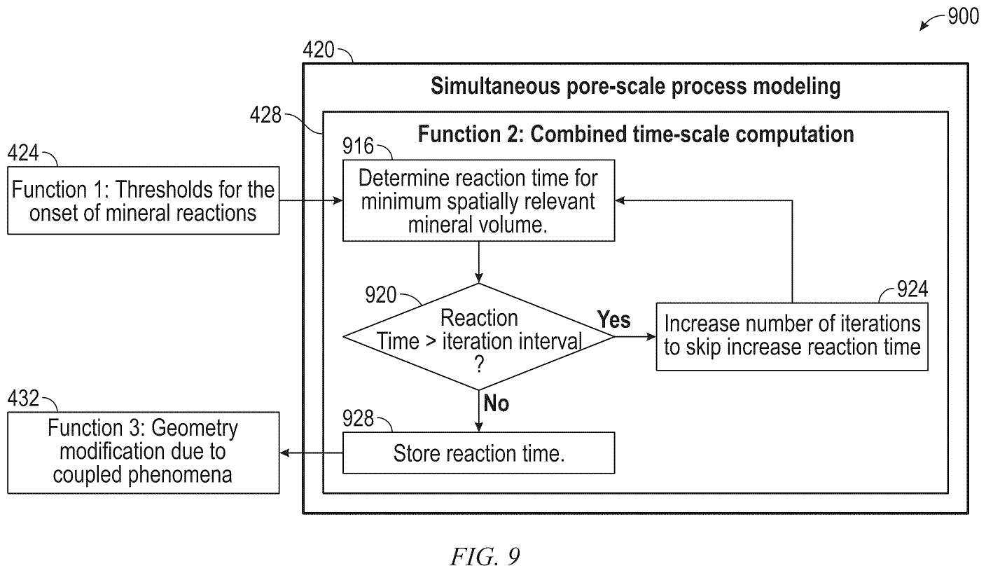

A check is then performed regarding the presence of a potential mineral process in each capillary (decision block 828). If the result of the check is YES, Function 2, combined time-scale computation, is performed (operation 428), as described more fully below in conjunction with FIG. 9; otherwise (NO branch of decision block 828; meaning that no presence of a pore-scale process was detected and the capillary dimensions remain constant within the time step), Function 2 and Function 3 in FIG. 4 (operations 428 and 432) are skipped, and the method 800 proceeds with operation 436 in FIG. 4 (‘capillary network update and time iteration’) where a new network is created, maintaining the same diameter values for the capillaries in following this branch of decision block 828. The existence of a potential onset of pore-scale processes is determined by thresholds from the literature. In physical processes, the thresholds are defined by particle dynamics expressions and, in chemical processes, by affinity models of surface-controlled crystallization. Operation 436 modifies the geometry of the capillaries that reach the thresholds for physical or chemical pore-scale processes (see the description above regarding operation 416); otherwise, the geometry of the capillaries remains constant during the iteration.

FIG. 9 is a flowchart of an example method 900 for computing the combined time-scale for the simulation, in accordance with an example embodiment. In one example embodiment, once Function 1 is performed and the thresholds for the onset of mineral reactions are determined (operation 424), Function 2, the computation of the combined time-scale, is performed (operation 428) on those capillaries encountered in the YES branch of 828 where the onset of at least one mineral reaction is detected. For each potential process determined as being present at each capillary within the capillary network model (CNM) geometry 232, an adequate time scale for pore-scale process simulation is computed. Employing estimates of the advection and diffusion time scales in the capillary, the reaction time required for minimum spatially relevant mineral volume accumulation is determined (operation 916). A check is then performed to determine if this minimum relevant reaction time is greater than the time interval considered in each iteration (decision block 920). If the reaction time is still larger than the iteration interval (YES branch of decision block 920), the number of iterations to skip is increased (operation 924); otherwise (NO branch of decision block 920), the current number of iterations adequate for the reaction time is stored (operation 928) and the geometry modification due to coupled phenomena, Function 3, is performed (operation 432). The pore-scale processes involved are geometry and flow dependent. Function 2 followed by Function 3 is performed, depending on the potential existence of a process within the capillary network. For example, when simulating a physical process with the known erosion and deposition law thresholds, the geometry variation of each capillary within the capillary network can be different since each capillary has different flow characteristics (i.e., flow rate and pressure gradient) that may lead to either erosion, deposition, both or none of those processes (see more details of such phenomena in the discussion of FIG. 4 and operation 716 of FIG. 7). When no process is present, the method skips Function 3 for that capillary since the diameter remains constant.

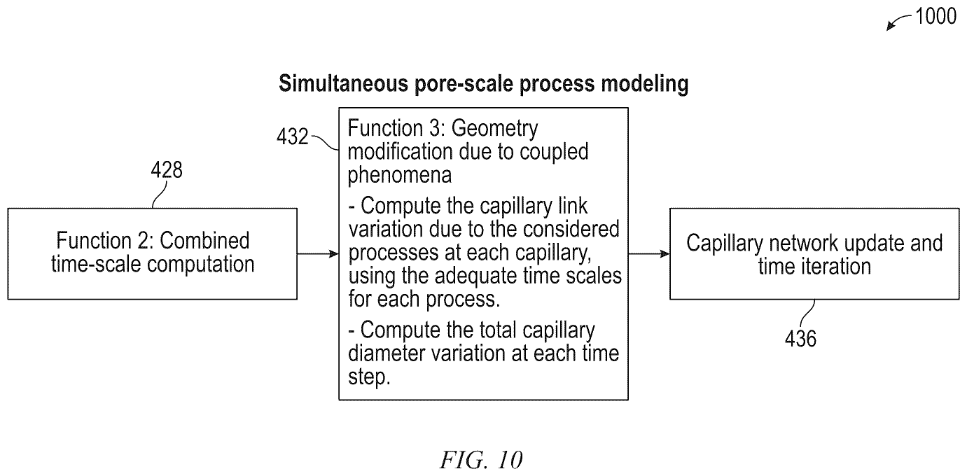

FIG. 10 is a flowchart 1000 of an example method for generating the geometry modification due to coupled phenomena, in accordance with an example embodiment. In one example embodiment, following the performance of Function 1, determining thresholds for the onset of mineral reactions (operation 424, FIG. 9), Function 2, the computation of the combined time-scale, is performed (operation 428); and Function 3, the generation of the geometry modification due to coupled phenomena, is performed (operation 432). In particular, the simultaneous mineral processes correlation is used for geometry evolution at each time step of pore-scale process simulations: once the flow steady state is reached, and the potential pore-scale processes are determined within the capillary network model (CNM) geometry 232, the capillary diameter variation due to the considered processes at each capillary is computed using the adequate time scales for each process, and the total capillary diameter variation at each time step is determined from the aggregate of all changes. Processes requiring more than one iteration to produce spatially relevant mineral volume as determined during operation 428 may not be computed in the current iteration. For example, the processes that increase the capillary diameter, such as erosion, dissolution, and the like, and the processes that reduce the link diameter, such as deposition, precipitation, and the like, are evaluated. The updates to the geometry of the capillary network are determined and passed on to operation 436 for the creation of a new capillary network to represent the digital version of the rock with modified dimensions. The generation of the geometry modification due to coupled phenomena is then performed (operation 436).

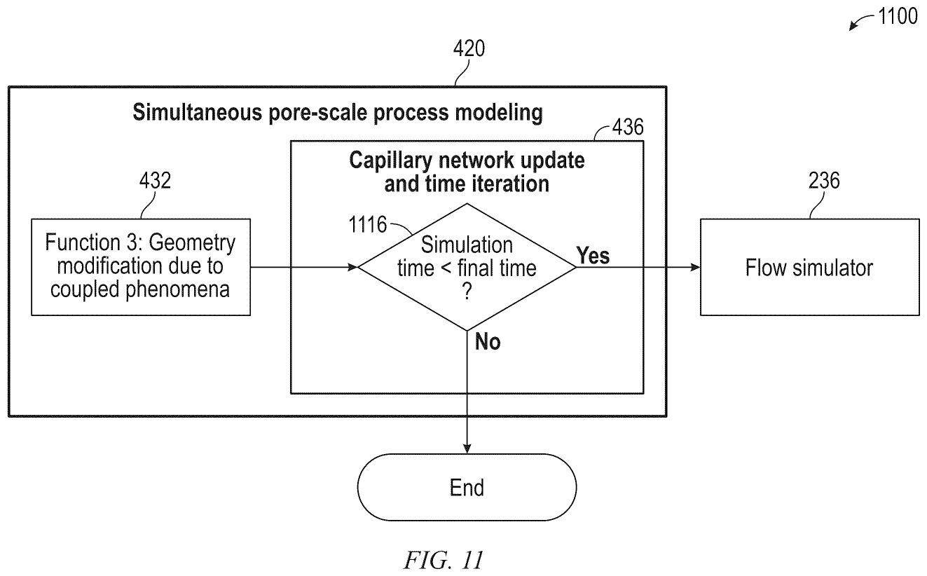

FIG. 11 is a flowchart 1100 of an example method for determining a completion time of a mineralization simulation, in accordance with an example embodiment. In one example embodiment, Function 3, the generation of the geometry modification due to coupled phenomena, is performed (operation 432). The geometry of the capillary network is updated to represent the digital rock with modified dimensions (operation 436). A check is performed to determine if the simulation is complete (the final time is greater than the simulation time) (decision block 1116). For example, if the sample being simulated has reached a specified level of porosity (NO branch of decision block 1116), the method 1100 ends; otherwise (YES branch of decision block 1116), the flow simulator 236 is run on the new porous geometry represented by the updated capillary network to obtain the pressure and velocity fields that serve as input for the next time step. When the end of the simulation time is reached, the final capillary network is updated. The geometry evolution data is stored for each time step of the simulation.

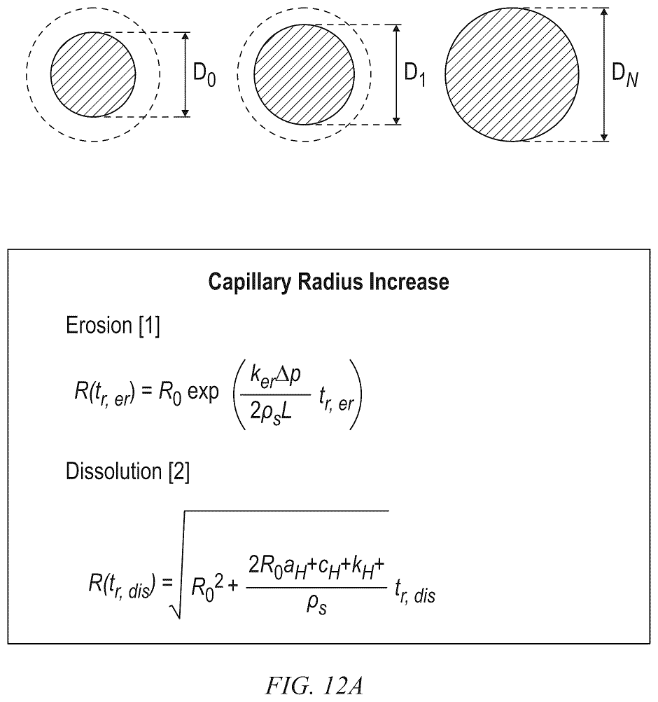

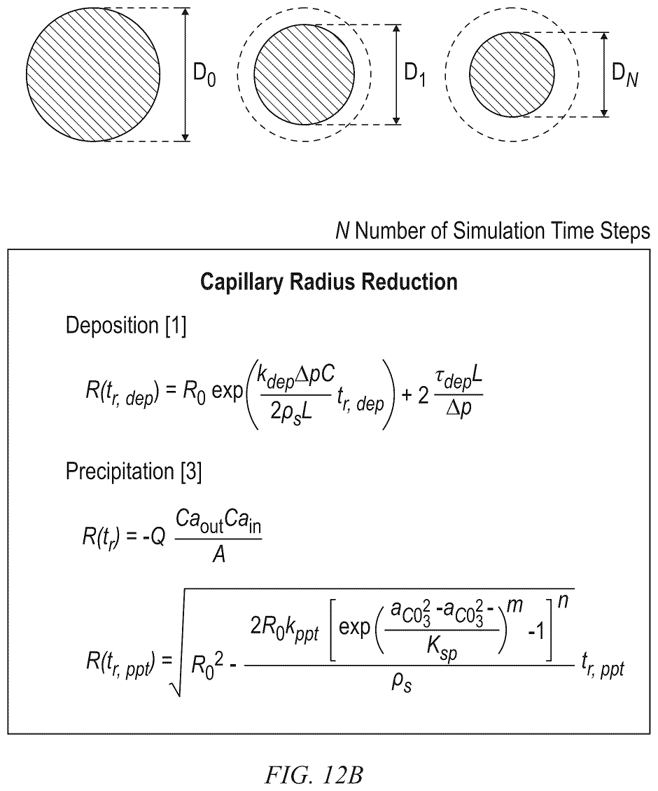

FIG. 12A illustrates the mathematical equations for quantifying the capillary radius increase resulting from erosion and dissolution, in accordance with an example embodiment. FIG. 12B illustrates the mathematical equations for quantifying the capillary radius reduction resulting from deposition and precipitation, in accordance with an example embodiment. All variables and constants considered in the formulations are collected and explained in the table of FIG. 13. These exemplary formulations were extracted from the literature.

FIG. 13 illustrate the parameters and variables of the simultaneous pore-scale processes, in accordance with an example embodiment. Such parameters are classified into those required as input for running the flow simulator and those required to simulate pore-scale reactions leading to geometry modification or GMM.

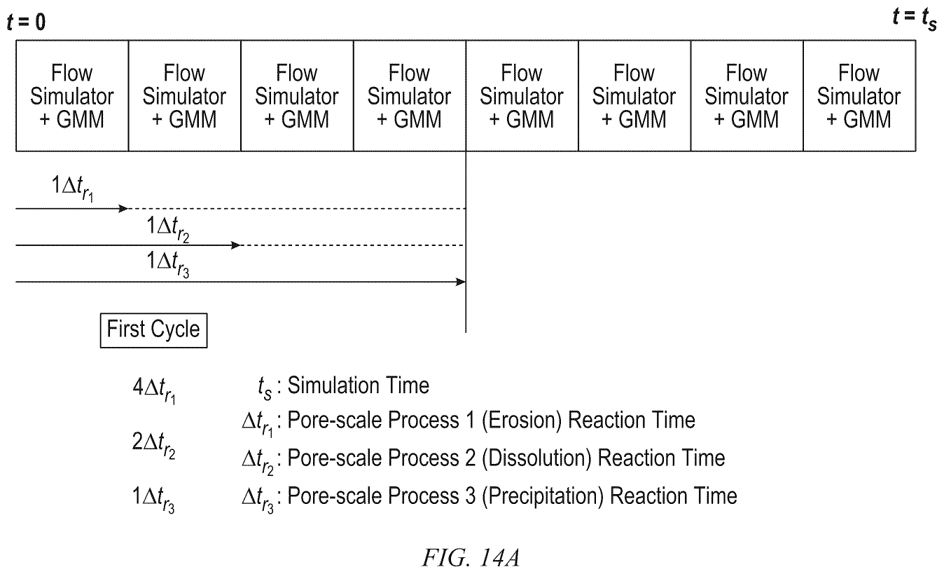

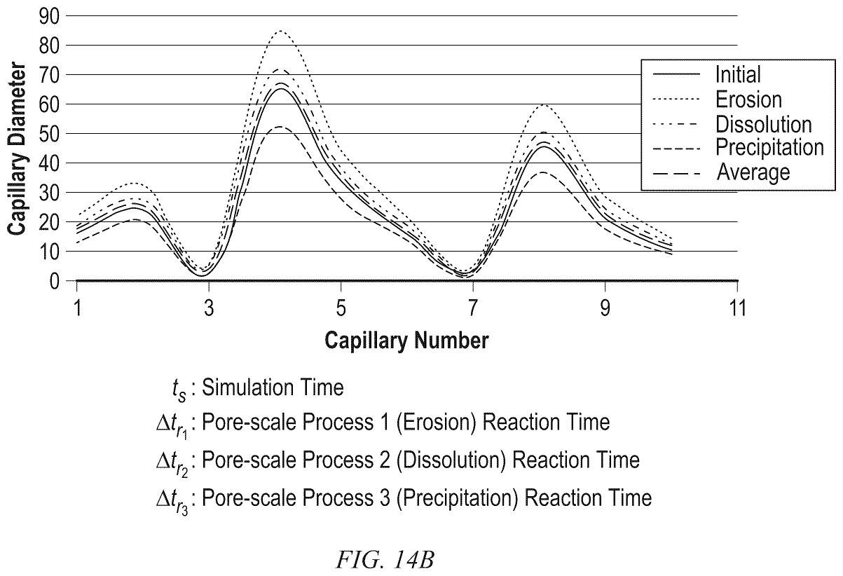

FIG. 14A illustrates a timing diagram for performing a mineralization simulation, in accordance with an example embodiment. During a time period between t=0 and t=tₛ, eight iterations through operations 416-436 are performed. Thus, the flow simulator 236 and the mineralization simulator 244 perform eight simulations. As illustrated in FIG. 14A, different effects are computed at different frequencies. For example, the first effect, erosion, is performed every simulation; the second effect, dissolution, is performed every other simulation; and the third effect, precipitation, is performed once every fourth simulation.

FIG. 14B is an illustrative graph of the diameter of each capillary, in accordance with an example embodiment. Deposition and mineral precipitation may partially or entirely clog pores and throats in the porous medium and change pore size distribution. As illustrated in FIG. 14B, the diameters of the capillaries vary widely. The horizontal axis of FIG. 14B shows the number of capillaries influenced by a capillary-dependent average evolution during simultaneous pore-scale processes. When simulating simultaneous reactions, the effect of the contribution of each process reaction on the average diameter variation of a rock sample 216 depends on several factors, including the variation of flow conditions in time (i.e., pressure and flow rate) and the magnitude of the contribution of each process parameter. In sum, solid particle erosion and mineral dissolution produce positive feedbacks on the local geometry variation, while solid particle deposition and mineral precipitation generate negative feedbacks. In consequence, the local average geometry evolution (i.e., of each capillary within the network) may be positive or negative during each iteration, leading to a capillary-dependent geometry and flow evolution.

In one aspect, an exemplary method includes, in response to receiving information including geometry of a capillary network representation of a porous rock sample, determining, using materials and selected reactions, a set of characteristics of the rock sample including phasic properties, flow conditions, and mineral reaction parameters; performing fluid-flow numerical simulations on the capillary network representation until reaching a steady state; computing mineral reaction rates, including physical rates and chemical rates, per capillary in the capillary network representation; and modeling simultaneous pore-scale processes to form a set of simulations. The modeling of the simultaneous pore-scale processes is done, for example, by: identifying, using predetermined known thresholds, existence of a pore-scale process at each capillary; determining a most suitable combined reaction time for simulations in the set of simulations using predetermined criteria and distinct time scales of a respective process per capillary; in response to receiving information for a respective capillary including time intervals and reaction rates, computing a change in diameter of each respective capillary; and updating geometry of the capillary network representation iteratively. Further steps include, in response to a determination the time of simulation is less than a final time, returning to perform addition flow simulations; in response to a determination the time of simulation is until a time of simulation is reached, updating a final capillary network representation; and storing geometry evolution data for each time step of simulations.

Note that blocks, modules, and the like, if not described in detail, can be implemented by the skilled artisan by adapting known systems and techniques in an implementation with software running on a general purpose computer.

Given the discussion thus far, it will be appreciated that, in general terms, an exemplary method, according to an aspect of the invention, includes the operations of obtaining a grey-scale tomography 224 of a sample 216 (operation 704); producing, using a capillary network extractor 228, a capillary network model (CNM) geometry 232 of the sample 216 based on the grey-scale tomography 224 (operation 404); performing, using a flow simulator 236, a fluid flow simulation to generate flow property fields 240 based on the capillary network model (CNM) geometry 232; and performing a mineralization simulation influenced by results of the flow property fields 240 to generate a modified capillary geometry 248 that represents the capillary network model (CNM) geometry 232 at a future point in time (operation 432).

In one example embodiment, the performing of the mineralization simulation further comprises computing mineral reaction rates per capillary (operation 416), where the computation of mineral reaction rates per capillary comprises incorporating an influence of local fluid flow conditions in a reaction rate.

In one example embodiment, the performing of the mineralization simulation further comprises computing mineral reaction rates per capillary (operation 416), where the computation of mineral reaction rates per capillary comprises identifying an occurrence, at a corresponding time interval, of one or more pore-scale processes at each capillary depending on predefined thresholds and on process-specific time-scales.

In one example embodiment, the performing of the mineralization simulation further comprises computing a change in diameter of each capillary and updating a geometry of the capillary network model (CNM) geometry 232.

In one example embodiment, the sample 216 is scanned, using a computed tomography (CT) scanner 220, to generate the grey-scale tomography 224.

In one example embodiment, subsurface carbon dioxide (CO₂) injection and sequestration in the sample 216 is determined based on the capillary network model (CNM) geometry 232.

In one example embodiment, a volume of carbon dioxide (CO₂) converted and stored in the sample 216 is estimated based on the capillary network model (CNM) geometry 232.

In one example embodiment, image processing is performed to convert a grey-scale of the grey-scale tomography 224 to binary values.

In one example embodiment, the performing of the mineralization simulation further comprises determining initial and boundary conditions and pore-scale parameters (operation 408).

In one example embodiment, the determining of the initial and boundary conditions and pore-scale parameters comprises determining, based on materials and selected reactions, phasic properties, flow conditions, and mineral reaction parameters (operation 408).

In one example embodiment, the determining of the initial and boundary conditions and pore-scale parameters further comprises imposing parameters related to the phasic properties and fluid flow as the initial conditions, setting parameters of liquid and solid phasic properties (operation 708), and setting parameters of pore-scale processes (operation 716).

In one example embodiment, the performing of the mineralization simulation further comprises determining one or more thresholds for an onset of one or more mineral reactions (operation 424); and determining a reaction time period for each mineral reaction for updating the capillary network model (CNM) geometry 232 and performing another flow simulation based on distinct time scales of a corresponding process per capillary (operation 428).

In one example embodiment, the performing of the fluid flow simulation and the performing of the mineralization simulation operations are repeated until a porosity of the sample 216 reaches a given level.

In one example embodiment, the performing of the mineralization simulation further comprises determining, at each iteration of the method, which processes of the mineralization simulation to compute based on a characteristic reaction time of the corresponding process.

In one example embodiment, a time scale of each process is determined within the capillary network model (CNM) geometry 232 by computing the characteristic reaction time required for minimum spatially relevant spatial variation due to a pore-scale process (operation 916).

In one example embodiment, porosity, permeability, and an amount of carbon dioxide precipitated and stored in the sample 216 is computed based on the capillary network model (CNM) geometry 232.

In one aspect, a computer program product comprises one or more tangible computer-readable storage media and program instructions stored on at least one of the one or more tangible computer-readable storage media, the program instructions executable by a processor, the program instructions comprising obtaining a grey-scale tomography 224 of a sample 216 (operation 704); producing, using a capillary network extractor 228, a capillary network model (CNM) geometry 232 of the sample 216 based on the grey-scale tomography 224 (operation 404); performing, using a flow simulator 236, a fluid flow simulation to generate flow property fields 240 based on the capillary network model (CNM) geometry 232; and performing a mineralization simulation influenced by results of the flow property fields 240 to generate a modified capillary geometry 248 that represents the capillary network model (CNM) geometry 232 at a future point in time (operation 432).

In one aspect, an apparatus comprises a memory and at least one processor, coupled to the memory, and operative to perform operations comprising obtaining a grey-scale tomography 224 of a sample 216 (operation 704); producing, using a capillary network extractor 228, a capillary network model (CNM) geometry 232 of the sample 216 based on the grey-scale tomography 224 (operation 404); performing, using a flow simulator 236, a fluid flow simulation to generate flow property fields 240 based on the capillary network model (CNM) geometry 232; and performing a mineralization simulation influenced by results of the flow property fields 240 to generate a modified capillary geometry 248 that represents the capillary network model (CNM) geometry 232 at a future point in time (operation 432).

In one example embodiment, a computational workflow enables a prediction, within a capillary network representation framework of the sample, of the spatiotemporal geometry evolution of the porous structure resulting from pore-scale processes.

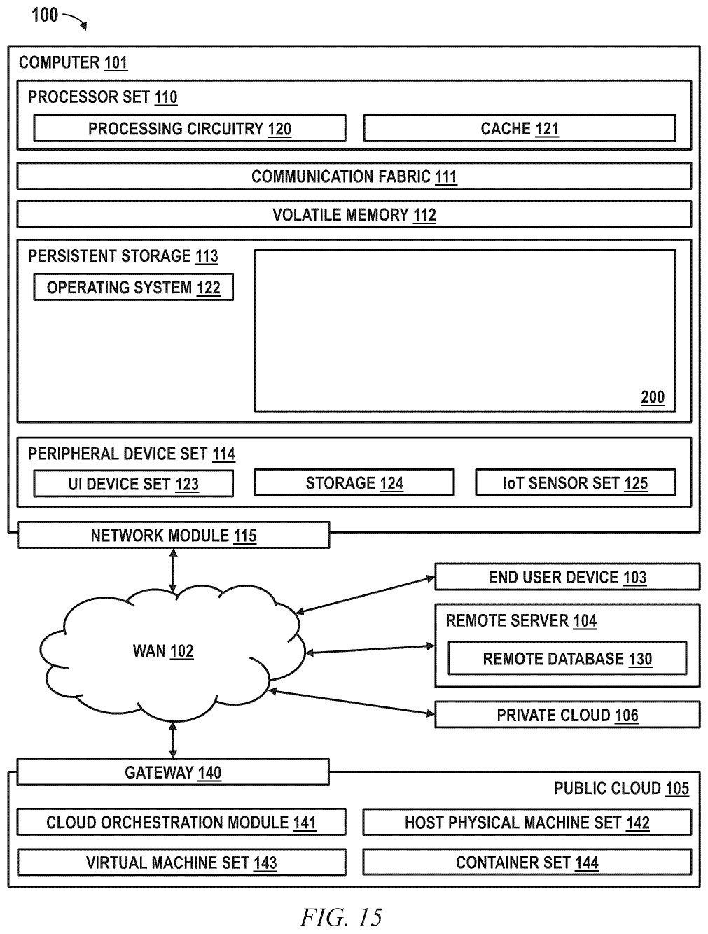

Refer now to FIG. 15.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as mineralization simulation system 200. Further, one or more embodiments, as noted, can improve a variety of practical applications such as reservoir engineering (a branch of petroleum engineering that applies scientific principles to the fluid flow through a porous medium during the development and production of oil and gas reservoirs so as to obtain a high economic recovery), oil recovery, and/or carbon dioxide geological sequestration. Thus, based on simulations herein, control signals could be sent (e.g., over WAN 102) to operate an oil field (drills, valves, or other oil field equipment, etc.) (e.g., in accordance with reservoir engineering principles), sequester carbon dioxide with carbon dioxide sequestration equipment, or the like. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
obtaining a grey-scale tomography of a sample;
producing, using a capillary network extractor, a capillary network model (CNM) geometry of the sample based on the grey-scale tomography;
performing, using a flow simulator, a fluid flow simulation to generate flow property fields based on the capillary network model (CNM) geometry; and
performing a mineralization simulation influenced by results of the flow property fields to generate a modified capillary geometry that represents the capillary network model (CNM) geometry at a future point in time.

2. The method of claim 1, wherein the performing of the mineralization simulation further comprises computing mineral reaction rates per capillary, where the computation of mineral reaction rates per capillary comprises incorporating an influence of local fluid flow conditions in a reaction rate.

3. The method of claim 1, wherein the performing of the mineralization simulation further comprises computing mineral reaction rates per capillary, where the computation of mineral reaction rates per capillary comprises identifying an occurrence, at a corresponding time interval, of one or more pore-scale processes at each capillary depending on predefined thresholds and on process-specific time-scales.

4. The method of claim 1, wherein the performing of the mineralization simulation further comprises computing a change in diameter of each capillary and updating a geometry of the capillary network model (CNM) geometry.

5. The method of claim 1, further comprising scanning, using a computed tomography (CT) scanner, the sample to generate the grey-scale tomography.

6. The method of claim 1, further comprising determining subsurface carbon dioxide (CO₂) injection and sequestration in the sample based on the capillary network model (CNM) geometry.

7. The method of claim 1, further comprising estimating a volume of carbon dioxide (CO₂) converted and stored in the sample based on the capillary network model (CNM) geometry.

8. The method of claim 1, further comprising performing image processing to convert a grey-scale of the grey-scale tomography to binary values.

9. The method of claim 1, wherein the performing of the mineralization simulation further comprises determining initial and boundary conditions and pore-scale parameters.

10. The method of claim 9, wherein the determining of the initial and boundary conditions and pore-scale parameters comprises determining, based on materials and selected reactions, phasic properties, flow conditions, and mineral reaction parameters.

11. The method of claim 10, wherein the determining of the initial and boundary conditions and pore-scale parameters further comprises imposing parameters related to the phasic properties and fluid flow as the initial conditions, setting parameters of liquid and solid phasic properties, and setting parameters of pore-scale processes.

12. The method of claim 1, wherein the performing of the mineralization simulation further comprises:
determining one or more thresholds for an onset of one or more mineral reactions; and
determining a reaction time period for each mineral reaction for updating the capillary network model (CNM) geometry and performing another flow simulation based on distinct time scales of a corresponding process per capillary.

13. The method of claim 1, further comprising repeating the performing of the fluid flow simulation and the performing of the mineralization simulation operations until a porosity of the sample reaches a given level.

14. The method of claim 1, wherein the performing of the mineralization simulation further comprises determining, at each iteration of the method, which processes of the mineralization simulation to compute based on a characteristic reaction time of the corresponding process.

15. The method of claim 14, wherein a time scale of each process is determined within the capillary network model (CNM) geometry by computing the characteristic reaction time required for minimum spatially relevant spatial variation due to a pore-scale process.

16. The method of claim 1, further comprising computing porosity, permeability, and an amount of carbon dioxide precipitated and stored in the sample based on the capillary network model (CNM) geometry.

17. A computer program product, comprising:
one or more tangible computer-readable storage media and program instructions stored on at least one of the one or more tangible computer-readable storage media, the program instructions executable by a processor, the program instructions comprising:
obtaining a grey-scale tomography of a sample;
producing, using a capillary network extractor, a capillary network model (CNM) geometry of the sample based on the grey-scale tomography;
performing, using a flow simulator, a fluid flow simulation to generate flow property fields based on the capillary network model (CNM) geometry; and
performing a mineralization simulation influenced by results of the flow property fields to generate a modified capillary geometry that represents the capillary network model (CNM) geometry at a future point in time.

18. The computer program product of claim 17, wherein the performing of the mineralization simulation further comprises computing mineral reaction rates per capillary, where the computation of mineral reaction rates per capillary comprises incorporating an influence of local fluid flow conditions in a reaction rate.

19. The computer program product of claim 17, wherein a computational workflow enables a prediction, within a capillary network representation framework of the sample, of the spatiotemporal geometry evolution of the porous structure resulting from pore-scale processes.

20. A system comprising:
a memory; and
at least one processor, coupled to said memory, and operative to perform operations comprising:
obtaining a grey-scale tomography of a sample;
producing, using a capillary network extractor, a capillary network model (CNM) geometry of the sample based on the grey-scale tomography;
performing, using a flow simulator, a fluid flow simulation to generate flow property fields based on the capillary network model (CNM) geometry; and
performing a mineralization simulation influenced by results of the flow property fields to generate a modified capillary geometry that represents the capillary network model (CNM) geometry at a future point in time.

21. The system of claim 20, wherein the performing of the mineralization simulation further comprises computing mineral reaction rates per capillary, where the computation of mineral reaction rates per capillary comprises incorporating an influence of local fluid flow conditions in a reaction rate.

22. The system of claim 20, wherein a computational workflow enables a prediction, within a capillary network representation framework of the sample, of the spatiotemporal geometry evolution of the porous structure resulting from pore-scale processes.

23. The system of claim 20, wherein the performing of the mineralization simulation further comprises computing mineral reaction rates per capillary, where the computation of mineral reaction rates per capillary comprises identifying an occurrence, at a corresponding time interval, of one or more pore-scale processes at each capillary depending on predefined thresholds and on process-specific time-scales.

24. The system of claim 20, wherein the performing of the mineralization simulation further comprises computing a change in diameter of each capillary and updating a geometry of the capillary network model (CNM) geometry.

25. The system of claim 20, wherein the at least one processor is further operative to perform image processing to convert a grey-scale of the grey-scale tomography to binary values.
